(19) United States
(12) Patent Application Publication
(10) Pub. No.: US 2023/0233226 A1
(43) Pub. Date: Jul. 27, 2023

(54) Surgical Tool with Retractable Blade and Bougie Passage and a Method of Use Thereof

(71) Applicants: Burton Bjorn Abbott, Winnipeg (CA); Ian Stuart Burron, Winnipeg (CA)

(72) Inventors: Burton Bjorn Abbott, Winnipeg (CA); Ian Stuart Burron, Winnipeg (CA)

(21) Appl. No.: 18/002,884

(22) PCT Filed: Jun. 21, 2021
(86) PCT No.: PCT/CA2021/050843

§ 371 Date: Dec. 22, 2022

Related U.S. Application Data
(60) Provisional application No. 63/043,930, filed on Jun. 25, 2020.

(51) Int. Cl. A61B 17/3211; A61M 16/04

(52) U.S. Cl. CPC A61B 17/3211; A61M 16/0472; A61B 2017/32113; A61M 2205/582

(57) ABSTRACT

A surgical tool has an elongate tool body that supports a retractable scalpel blade at a working end of the tool body. A bougie passage extends through the tool body to receive a bougie slidable longitudinally through the passage in the tool body. The tool body may be a single scalpel handle, or a pair of forceps. In either instance, the tool may be used to perform a cricothyrotomy procedure on patient or introduce a thoracostomy tube into a patient. In each instance, the scalpel blade forms an incision, followed by retraction of the blade and insertion of the bougie while the tool remains inserted into the patient. After removal of the tool, the bougie is used to guide subsequent insertion of a tube into the patient.

### FIELD OF THE INVENTION

The present invention relates to a surgical tool, for example a surgical scalpel or surgical forceps, in which the surgical tool is provided with a scalpel blade operable between a working position for cutting and a stored position in which the cutting edge of the scalpel blade is at least partly covered. More particularly, the present invention relates to a surgical tool having a scalpel blade and a bougie passage extending through a tool body of the surgical tool for insertion of a bougie therethrough, for example, for insertion of a bougie for guiding an endotracheal tube in a cricothyrotomy procedure performed with the surgical tool or for insertion of a bougie for guiding a thoracostomy tube in a thoracostomy procedure performed with the surgical tool.

### BACKGROUND

Cricothyrotomy is an emergency procedure used to intubate patients when standard intubation procedures have failed or are not possible. This procedure involves placing an endotracheal tube through the cricothyroid membrane into the trachea. All emergency departments make provisions for emergency cricothyrotomy, typically according to one of the following procedures.

Cricothyrotomy procedure A. The most common procedure involves placing a needle on a syringe through the cricothyroid membrane, remove the syringe, introduce a guidewire through the needle through the cricothyroid membrane and into the trachea, remove the needle which leaves the guidewire through the cricothyroid membrane and in the trachea, cut alongside the guidewire with a scalpel, introduce an endotracheal tube/attached dilator over the guidewire, remove the guidewire/dilator. The endotracheal tube is left in place and used to ventilate the patient.

Cricothyrotomy procedure B. The above Procedure A has been criticized for being overly cumbersome and too time consuming to carry out during an airway crisis. The current generally recommended procedure, according to many emergency medicine authorities, is to incise the skin vertically over the cricothyroid membrane, incise the membrane itself horizontally, insert finger through the cricothyroid membrane, remove scalpel, insert bougie through the cricothyroid membrane alongside finger, insert endotracheal tube through the cricothyroid membrane and into the trachea—using the bougie as a guide. There is still concern that using Procedure B involves placing one's finger in close proximity to the scalpel blade (risk for injury) and/or involves entering and exiting the cricothyroid membrane with various items which increases the risk of a false tract. Such risk would be greatest among those with neck obesity since the pathway from the skin surface into the airway is long and could become lost. Such misplacement of the endotracheal tube would lead to a catastrophic airway failure.

Thoracostomy tube (chest tube) insertion is another procedure performed in emergency situations. In this instance, a tube is inserted into the thorax, into the pleural space between ribs on the lateral chest wall. The tube is inserted to remove air and/or fluid (blood, pus, fluid) from the inside of the chest to allow lung expansion. The standard procedure involves cutting and dissecting through the chest wall and then entering into the pleural space with a closed clamp. The clamp is then opened—essentially tearing a larger hole into the pleural space. The physician then places a finger into the pleural space, verifying correct position. A thoracostomy tube is then inserted into the pleural space to allow drainage.

A common and potentially dangerous complication of this procedure is the formation of a “false tract” that doesn't actually lead into the pleural space. Particularly in an obese patient, tubes have been misplaced in the chest wall rather than the pleural space. The primary reason for this misplacement, is that items (i.e. scalpel, clamp, finger) enter and exit the chest wall and it is possible for the physician to lose the correct pathway back into the pleural space, thereby placing the thoracostomy tube into a false tract rather than in the pleural space.

One proposed technique has been to use a bougie as a guide but this still requires handling multiple instruments and requires items entering and exiting the pleural space, increasing the risk for blind tract formation.

### SUMMARY OF THE INVENTION

According to one aspect of the invention there is provided a surgical tool for use with a bougie, the tool comprising:

a tool body which is elongate in a longitudinal direction between a working end and a handle end;

a scalpel blade supported on the working end of the tool body so as to be operable between a working position in which a cutting edge of the scalpel blade is exposed for cutting and a stored position in which the cutting edge of the scalpel blade is at least partly covered;

a bougie passage formed on the tool body to extend in the longitudinal direction between opposing open ends of the bougie passage in proximity to the working end and the handle end of the tool body respectively, the bougie passage being arranged to receive the bougie slidably in the longitudinal direction therethrough.

In the preferred embodiment, the scalpel blade is supported on the tool body to be longitudinally slidable relative to the tool body between the working position and the stored position thereof.

Preferably, one of the open ends of the bougie passage is located at the working end of the tool body, and one of the open ends of the bougie passage is located at the handle end of the tool body such that the bougie passage spans a full length of the tool body.

In one embodiment, the tool may be a scalpel in which the tool body consists of a single elongated scalpel handle.

In this instance, another aspect of the invention relates to the use of the tool to perform a cricothyrotomy procedure on a patient.

More particularly, according to another aspect of the present invention there is provided a method of using the tool to perform a cricothyrotomy procedure on a patient, the method including:

using the scalpel blade in the working position to incise a cricothyroid membrane of the patient;

displacing the scalpel blade into the stored position while keeping the tool body in place through the cricothyroid membrane of the patient;

inserting the bougie through the bougie passage in the tool body such that the bougie is directed into a trachea of the patient;

removing the tool from the patient while keeping the bougie directed into the trachea of the patient;

introducing an endotracheal tube into the trachea of the patient over the bougie; and

removing the bougie from the patient while keeping the endotracheal tube directed into the trachea of the patient.

In one example, the surgical tool may be provided as part of a kit used for emergency cricothyrotomy. This kit consists of the following items: (i) Bougie, (ii) 6 mm internal diameter endotracheal tube; and (iii) The surgical tool according to the present invention in the form of a firm plastic scalpel with a retractable blade with a channel to accommodate the bougie in which the channel runs the length of the scalpel.

In this example, the cricothyrotomy kit would be used as follows for cricothyrotomy: (i) Use the scalpel to incise vertically over the cricothyroid membrane, (ii) Incise the cricothyroid membrane horizontally—cut a large enough opening to allow introduction of a 6 mm internal diameter endotracheal tube, (iii) Retract the scalpel blade while keeping then end of the scalpel in place though the cricothyroid membrane, (iv) Direct the direct the scalpel in a caudad direction, (v) Introduce the bougie through the channel so that it is directed into the trachea, (vi) Confirm bougie position in the trachea with palpable clicks from the trachea rings and introduce the bougie to a predetermined depth (mark on bougie), (vii) Remove the scalpel, holding the bougie in place, (viii) Introduce 6 mm internal diameter endotracheal tube over the bougie, using the bougie as a guide, and (ix) Remove the bougie, ventilate though the endotracheal tube, and suture the endotracheal tube in place.

In another embodiment, the tool may be a pair of forceps in which the tool body consists of two members each extending longitudinally between a handle portion and a clamping portion, the two members being pivotally coupled to one another at respective intermediate locations on the members so as to be adapted to clamp an object between the clamping portions when the handle portions are displaced towards one another. Preferably, the scalpel blade is supported on one of the two members of the tool body and the bougie passage is formed on another one of the two members of the tool body.

In this instance, another aspect of the present invention relates to the use of the tool to introduce a thoracostomy tube into a patient.

More particularly, according to another aspect of the present invention there is provided a method of using the tool to introduce a thoracostomy tube into a patient at a landmarked site, the method comprising:

using the scalpel blade in the working position to incise into subcutaneous tissue at the landmarked site;

displacing the scalpel blade into the stored position while keeping the tool body in place at the landmarked site;

pushing the tool body into a pleural space of the patient;

inserting the bougie through the bougie passage in the tool body such that the bougie is directed into the pleural space;

removing the tool from the patient while keeping the bougie directed into the pleural space;

introducing a thoracostomy tube into the pleural space of the patient over the bougie; and

removing the bougie from the patient while keeping the thoracostomy tube directed into the pleural space of the patient.

In another example, the surgical tool may be provided as part of a kit used for thoracostomy tube insertion. This kit consists of the following items: (i) Bougie, (ii) a thoracostomy tube; and (iii) the surgical tool according to the present invention in the form of a multifunction clamp for medical procedures in which one arm of the clamp has a channel for introduction of a bougie and the other arm of the clamp has a small retractable scalpel blade for cutting through tissue.

In this example, the thoracostomy kit would be used as follows for thoracostomy tube introduction: (i) Landmark site for thoracostomy tube introduction, (ii) Infiltrate with local anesthetic, (iii) Incise with the scalpel blade, (iv) After cutting well into the subcutaneous tissue, retract the scalpel blade, then blunt dissect with the clamp and fingers, (v) Push the closed clamp into the pleural space while grasping near the end of the clamp so that the clamp penetrates approx. 1.5 cm, (vi) Open the clamp, tearing open a hole in the parietal pleura large enough to insert the physician's index finger, (vii) Close the clamp, (viii) While maintaining the position of the end of the closed clamp inside the pleural space, introduce the index finger to verify position within the pleural space, (ix) Direct the end of the forceps posteriorly inside the pleural space, (x) Direct the bougie down the channel and into the chest to a predetermined distance — marked on the bougie, (xi) Holding the bougie in place, slide the clamp out of the pleural space, (xii) Insert the thoracostomy tube into the pleural space using the bougie as a guide, (xiii) Remove the bougie, (xiv) Close the surrounding chest wall wound and suture the thoracostomy tube to the chest wall, and (xv) Attach the thoracostomy tube to drainage.

### BRIEF DESCRIPTION OF THE DRAWINGS

Various embodiments of the invention will now be described in conjunction with the accompanying drawings in which:

FIG. 1 is a perspective view of a first embodiment of the surgical tool in a working position of the blade;

FIG. 2 is a perspective view of the first embodiment of the surgical tool according to FIG. 1 in a stored position of the blade;

FIG. 3 is a sectional view along the line 3-3 in FIG. 2;

FIG. 4 is an end elevational view of the handle end of the first embodiment of the tool according to FIG. 1;

FIGS. 5 through 10 are schematic representations of various steps in a cricothyrotomy procedure using the surgical tool according to the first embodiment of FIG. 1;

FIG. 11 is a schematic view of a second embodiment of the surgical tool in a closed position and in a working position of the blade;

FIG. 12 is a schematic view of the surgical tool according to the second embodiment of FIG. 11 in an open position and in a stored position of the blade;

FIG. 13 is a sectional view along the line 13-13 in FIG. 12; and

FIGS. 14 through 19 are schematic representations of various steps in a thoracostomy tube insertion procedure using the surgical tool according to the second embodiment of FIG. 11.

In the drawings like characters of reference indicate corresponding parts in the different figures.

### DETAILED DESCRIPTION

Referring to the accompanying figures there is illustrated a surgical tool generally indicated by reference numeral 10. The tool 10 is useful for a variety of surgical procedures as described in the following. Although various embodiments are disclosed herein and illustrated in the accompanying figures, the common features of the various embodiments will first be described.

In each instance the surgical tool 10 generally includes a tool body 12 which is elongate in a longitudinal direction between a working end 14 and an opposing handle end 16. The handle end forms a handle that is suitable for gripping in a single hand of a user, whereas the working end supports a scalpel blade 18 thereon having a cutting edge suitable for performing incisions in surgical procedures.

In each instance, the blade 18 is supported on the body so as to be movable between (i) a working position in which the blade protrudes longitudinally outward beyond the end of the tool body such that the cutting edge of the blade is exposed and suitably oriented for cutting, and (ii) a stored position in which the blade is retracted longitudinally inwardly into the body such that the cutting edge is at least partially, and more preferably fully covered by the surrounding tool body.

The tool body 12 is further provided with a bougie passage 20 formed therein to extend longitudinally substantially the full length of the tool body in the longitudinal direction from a first end opening 22 at the working end of the tool body to a second end opening 24 at the handle end of the body. The bougie passage 20 is a tubular passage which is fully enclosed between the opposing first and second ends thereof and is suitably arranged to receive a bougie 40 longitudinally slidable therethrough.

In a typical surgical operation, the surgical tool 10 is primarily used for insertion of a tube 42 into a patient in accordance with various procedures. Typically, the scalpel blade is initially positioned in the working position to enable cutting through a tissue layer of the patient to provide access to a cavity within the body of the patient through the incision. While maintaining the working end of the tool body penetrated into the cavity in the patient, the scalpel is retracted so that an operator of the surgical tool can safely locate the working end of the surgical tool within the body of the patient using their finger without risk of cutting their finger on the retracted blade. The operator then inserts the bougie 40 into the second end opening 24 of the bougie passage 20 at the handle end of the tool such that the bougie can be displaced fully through the body and protrude from the first end opening 22 at the working end. When the end of the bougie has been properly located within the body of the patient, the surgical tool can be slidably removed from the bougie while maintaining the bougie in place within the patient. A suitable tube 42 can then be inserted into the patient over top of the bougie so that the bougie acts as a guide for guiding proper placement of the tube within the patient. While maintaining the tube in place within the patient, the bougie can then be removed.

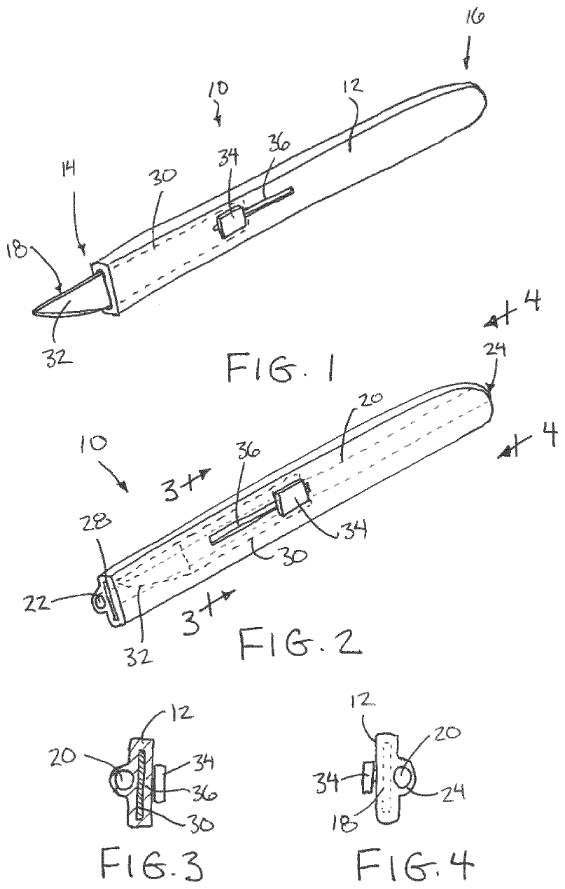

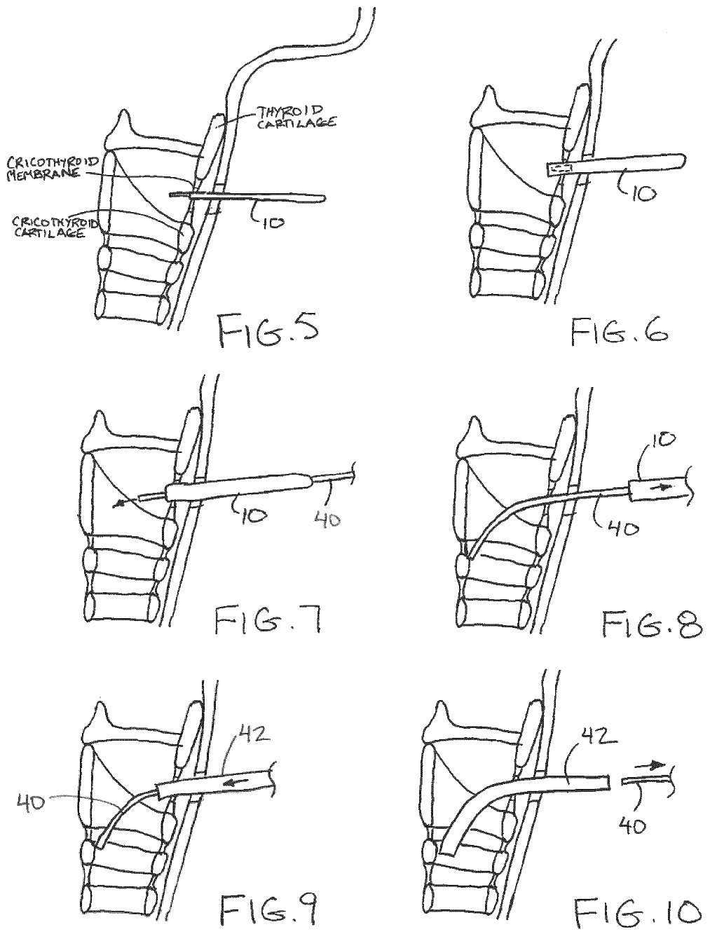

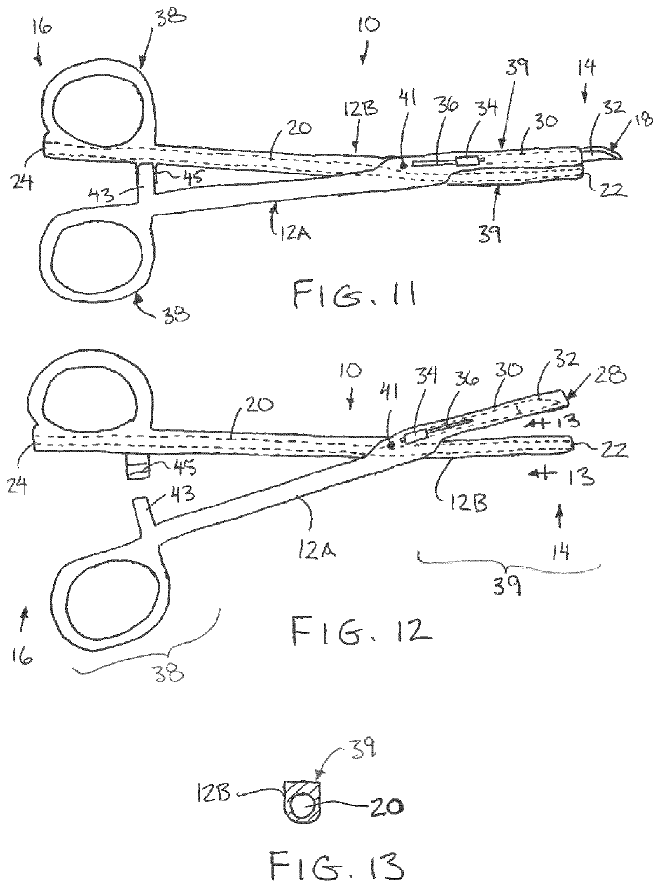

Turning now more particularly to the first embodiment illustrated in FIGS. 1 through 10, the surgical tool in this instance is a surgical scalpel in which the tool body 12 comprises a single member spanning the full length of the tool so as to be elongate in the longitudinal direction between the opposing working end and handle end of the body. A hollow cavity is provided internally within the body which is elongate within the longitudinal direction and which is in open communication with a blade opening 28 at the working end of the body such that the scalpel blade can be supported with in the cavity in the body to selectively protrude through the blade opening in the working position thereof. More particularly the blade includes (i) a mounting portion 30 which is mounted internally within the cavity in the tool body for sliding in the longitudinal direction relative to the tool body and (ii) a blade portion 32 that locates the cutting-edge thereon and which extends longitudinally from the mounting portion such that the blade portion protrude longitudinally outward beyond the end of the tool body through the blade opening in the working position while being retracted fully into the interior hollow cavity within the blade body in the stored position.

In order to control the positioning of the blade between the working and stored position thereof, an actuator button 34 is supported externally at one side of the tool body. The actuator button 34 is connected to the mounting portion 30 of the blade through an actuator slot 36 formed in the tool body. The actuator slot is elongate in the longitudinal direction of the tool body and communicates with the hollow cavity locating the mounting portion of the blade therein along the full length thereof. The actuator button is longitudinally slidable along the full length of the slot between opposing ends thereof corresponding to the blade being situated in the opposing working and stored positions thereof respectively.

The bougie passage 20 in this instance is a tubular passage with a circular cross-section which extends alongside the body so as to be fully enclosed between the opposing open ends 22 and 24. The bougie passage 20 is located along the side of the body which is laterally opposed from the side of the body locating the actuator button 34 thereon.

When the surgical tool 10 is used as a kit for emergency cricothyrotomy, the kit can include (i) the surgical tool according to the first embodiment of FIGS. 1 through 10 , (ii) a bougie 40 that can slidably pass through the bougie passage in the surgical tool, and (iii) an endotracheal tube having a 6 mm internal diameter that can pass slidably over top of the bougie. Use of the tool for cricothyrotomy begins with using the surgical tool with the blade in the working position to incise vertically over the cricothyroid membrane. The cricothyroid membrane is then incised horizontally to form a cut with a large enough opening to allow introduction of the endotracheal tube 42. The operator then retracts the blade into the stored position while keeping the working end of the tool body in place protruding through the cricothyroid membrane. The operator of the tool, that is the surgeon, directs the working end of the tool body in a caudad direction towards the head of the patient and then introduces the bougie 40 through the bougie passage 20 such that the inner end of the bougie is directed into the trachea of the patient. The surgeon can confirm proper placement of the bougie within the trachea by palpable clicks from the trachea rings, followed by the bougie being introduced to a predetermined depth, which may be marked on the bougie. The surgeon can then remove the surgical tool while holding the bougie in place within the patient. The endotracheal tube 42 with the 6 mm internal diameter is then placed over top of the bougie and inserted into the patient using the bougie as a guide. Once the endotracheal tube 42 has been properly placed, the bougie can be removed to enable ventilating through the endotracheal tube. The endotracheal tube is then sutured in place within the patient.

Turning now to a second embodiment of the surgical tool as illustrated in FIGS. 11 through 19, the surgical tool 10 in this instance comprises surgical forceps, also known as a surgical clamp. The tool body in this instance comprises two separate members comprising a first member 12A and a second member 12B. Each of the members extends from a handle portion 38 forming a finger receiving loop at the handle end of the body to a working portion 39 having an inner clamping face thereon at the opposing working end of the body. A pivot coupling 41 is provided at an intermediate location between the working end and the handle end on each member which pivotally couples the first and second members together in a manner similarly to a conventional pair of scissors or a set of pliers.

In this manner the first and second members of the tool body extend generally alongside one another in a clamped position as shown in FIG. 11 in which the inner clamping faces of the members are directly adjacent to one another for clamping an object therebetween while the handle portions 38 are similarly adjacent one another. The first and second members of the tool body can be pivoted relative to one another from the closed and clamped position of FIG. 11 into an open position as shown in FIG. 12 in which the clamping faces of the working portions 39 of the members are spaced apart from one another by displacing the finger loops formed at the handle portions 38 apart from one another. A hook 43 is provided at the inner side of one of the handle portions 38 for selectively engaging a catch 45 at the inner side of the other handle portion 38 to selectively and releasably latch the first and second members in the closed position of FIG. 12 when desired.

The working portion 39 of the first member 12A includes a hollow cavity therein in communication with a blade opening at the working end of the tool body to receive the mounting portion 30 of the blade therein such that the blade portion 32 can extend through the blade opening or be retracted into the hollow cavity as it is displaced between the working and stored positions thereof similarly to the previous embodiments. An actuator button 34 is connected to the mounting portion 30 of the blade through an actuator slot 36 also in a manner similar to the previous embodiment so as to enable the operator to displace the blade between the working and stored positions.

The second member 12B in this instance forms the bougie passage 20 therein in which the first end of the bougie passage is located at the working end of the second member of the tool body while the second end of the bougie passage is located at the handle end of the second member of the tool body. In this instance the bougie passage again extends the full length of the tool body while being fully enclosed between the opposing open ends thereof to allow passage of the bougie 40 longitudinally slidable therethrough. The bougie passage is offset from the pivot coupling 41 such that there is no interference with passage of the bougie through the bougie passage.

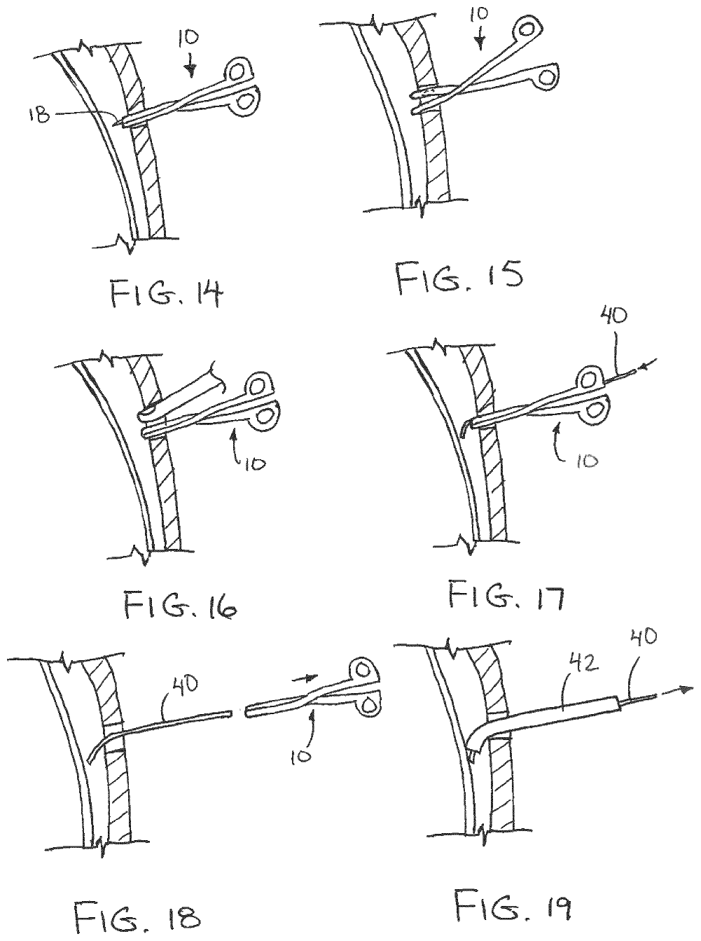

When the surgical tool 10 is used for thoracostomy tube insertion, the tool may be provided as a kit together with a suitable bougie 40 that is passable through the passage in the tool and a thoracostomy tube 42 that is slidable over top of the bougie 40. When using the surgical tool 10 according to the second embodiment of FIGS. 11 through 19 for thoracostomy tube introduction, the procedure is initiated by locating a landmark site for the tube introduction on the chest of the patient. Once the site has been infiltrated with local anaesthetic, the operator of the tool, that is the surgeon, uses the blade 18 of the tool in the working position to incise well into the subcutaneous tissue of the patient. The blade can then be retracted into the stored position followed by blunt dissection with the blunt end of the surgical tool and the fingers of the surgeon. The closed tool with the blade in the stored position can be pushed into the pleural space while grasping near the end of the clamp so that the working end of the surgical tool penetrates approximately 1.5 cm into the pleural space. As shown in FIG. 15, the tool can be opened to assist in tearing open a hole in the parietal pleura large enough to insert the surgeon's index finger. With the tool in the closed position, and while maintaining the position of the working end of the closed surgical tool inside the pleural space, the index finger of the surgeon can be introduced to verify the position of the end of the tool within the pleural space as shown in FIG. 16. The end of the surgical tool is then directed posteriorly inside the pleural space such that a bougie can be directed down the bougie passage of the tool and into the pleural space to a predetermined distance marked on the bougie as represented in FIG. 17. While holding the bougie in place, the surgical tool can be slidably removed from the pleural space as represented in FIG. 18. A thoracostomy tube 42 can then be introduced into the pleural space using the bougie 40 as a guide, followed by removal of the bougie as represented in FIG. 19. The surrounding chest wall wound can then be closed with the thoracostomy tube sutured to the chest wall, followed by attachment of the thoracostomy tube to drainage to complete the procedure.

Since various modifications can be made in my invention as herein above described, and many apparently widely different embodiments of same made, it is intended that all matter contained in the accompanying specification shall be interpreted as illustrative only and not in a limiting sense.

1. A surgical tool for use with a bougie, the tool comprising:
a tool body which is elongate in a longitudinal direction between a working end and a handle end;
a scalpel blade supported on the working end of the tool body so as to be operable between a working position in which a cutting edge of the scalpel blade is exposed for cutting and a stored position in which the cutting edge of the scalpel blade is at least partly covered;
a bougie passage formed on the tool body to extend in the longitudinal direction between opposing open ends of the bougie passage in proximity to the working end and the handle end of the tool body respectively, the bougie passage being arranged to receive the bougie slidably in the longitudinal direction therethrough.

2. The tool according to claim 1 wherein the scalpel blade is supported on the tool body to be longitudinally slidable relative to the tool body between the working position and the stored position thereof.

3. The tool according to claim 1 wherein one of the open ends of the bougie passage is located at the working end of the tool body.

4. The tool according to claim 1 wherein one of the open ends of the bougie passage is located at the handle end of the tool body.

5. The tool according to claim 1 wherein the bougie passage spans a full length of the tool body.

6. The tool according to claim 1 wherein the tool is a scalpel in which the tool body consists of a single elongated scalpel handle.

7. The tool according to claim 1 wherein the tool is a pair of forceps in which the tool body consists of two members each extending longitudinally between a handle portion and a clamping portion, the two members being pivotally coupled to one another at respective intermediate locations on the members so as to be adapted to clamp an object between the clamping portions when the handle portions are displaced towards one another.

8. The tool according to claim 7 wherein the scalpel blade is supported on one of the two members of the tool body and the bougie passage is formed on another one of the two members of the tool body.

9. Use of the tool according to claim 1 to perform a cricothyrotomy procedure on a patient.

10. A method of using the tool according to claim 1 to perform a cricothyrotomy procedure on a patient, the method including:
using the scalpel blade in the working position to incise a cricothyroid membrane of the patient;
displacing the scalpel blade into the stored position while keeping the tool body in place through the cricothyroid membrane of the patient;
inserting the bougie through the bougie passage in the tool body such that the bougie is directed into a trachea of the patient;
removing the tool from the patient while keeping the bougie directed into the trachea of the patient;
introducing an endotracheal tube into the trachea of the patient over the bougie; and
removing the bougie from the patient while keeping the endotracheal tube directed into the trachea of the patient.

11. Use of the tool according to claim 1 to introduce a thoracostomy tube into a patient.

12. A method of using the tool according to claim 1 to introduce a thoracostomy tube into a patient at a landmarked site, the method comprising:
using the scalpel blade in the working position to incise into subcutaneous tissue at the landmarked site;
displacing the scalpel blade into the stored position while keeping the tool body in place at the landmarked site;
pushing the tool body into a pleural space of the patient;
inserting the bougie through the bougie passage in the tool body such that the bougie is directed into the pleural space;
removing the tool from the patient while keeping the bougie directed into the pleural space;
introducing a thoracostomy tube into the pleural space of the patient over the bougie; and
removing the bougie from the patient while keeping the thoracostomy tube directed into the pleural space of the patient.
